(19) United States
(12) Patent Application Publication
(10) Pub. No.: US 2009/0246879 A1
(43) Pub. Date: Oct. 1, 2009

(54) Materials and Methods Relating to Nano-Tags and Nano-Brands

(75) Inventors: Radoje T. Drmanac, Palo Alto, CA (US); Brian Hauser, Campbell, CA (US); Joseph Komoski, Mission Viejo, CA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: CALLIDA GENOMICS, Sunnyvale, CA (US)

(21) Appl. No.: 12/414,208

(22) Filed: Mar. 30, 2009

Related U.S. Application Data
(63) Continuation of application No. 10/741,627, filed on Dec. 19, 2003.
(60) Provisional application No. 60/435,374, filed on Dec. 20, 2002.
(60) Provisional application No. 60/435,173, filed on Dec. 20, 2002.

(51) Int. Cl. G01N 33/00

(52) U.S. Cl. 436/89; 436/94

(57) ABSTRACT

The invention relates to novel methods and materials for encoding and decoding information on individual molecules through the use of nano-tags, nano-barcodes, or modifications of a native molecule.

### 1. CROSS-REFERENCE TO OTHER APPLICATIONS

This application claims the benefit of priority to U.S. Provisional Application Ser. No. 60/435,374 filed Dec. 20, 2002, entitled “Nano-tags and Nano-barcodes,” Attorney Docket No. 30311/39036 and U.S. Provisional Application Ser. No. 60/435,173 filed Dec. 20, 2002, entitled “Nano-tags and Nano-barcodes,” Attorney Docket No. 30311/39037. These and all other U.S. patents and patent applications are herein incorporated by reference in their entirety.

### 2. BACKGROUND

2.1 Field of the Invention

The invention relates to novel methods, materials and devices for encoding and decoding information on individual molecules through the use of nano-tags, nano-barcodes or modifications of a native molecule.

2.2 Background of the Invention

The use of arrays or encoded microparticles is an important method of achieving multiplex biological or chemical assays in a single reaction. The use of such techniques for DNA sequencing by hybridization (SBH) was originally proposed by Drmanac et al., U.S. Pat. No. 5,202,231 (herein incorporated by reference in its entirety). Array technology has been developed in which thousands of different nucleic acid “probe” molecules can be tested simultaneously. Likewise, commercial examples of encoded micro-particles include Luminex™ beads, which are available in hundreds of distinct formulations, and Surromed™ nano-barcodes, which are created from unique metallic micro-strips containing a complex pattern of visually distinguishable metal bands.

Most modern encoding techniques are based on a unique chemical or physical attribute. In array technology, any one particular probe is presented as a population of many individuals within a spot on an array and encoded by physical positioning. In micro-particle technology, any one particular probe is presented as a population of many individuals attached to the surface of a microparticle and encoded and detected by the presence of another molecule such as a covalently attached fluorophore. Thus, all current biopolymer sequencing technologies require multiple copies of the sample to generate adequate signal. One immediate disadvantage to this method is the need for literally thousands of successful events to build sufficient signal above background for any one probe. Hence, arrays and encoded particles are relatively large in size in order to detect the code or signal from an assay. Furthermore, arrays and microparticles have synthesis requirements that limit the use of combinatorial chemistry procedures, such as mix-and-divide synthesis. As a result, the potential to encode millions of individual particles is greatly complicated by the need to handle each reaction individually. The present invention solves this limitation and allows for the detection of individually nano-tagged molecules which allows one to both encode and decode information about that molecule.

Nano-tags are any nanometer-scale molecular structure that possesses chemical or physical attributes allowing facile and individual molecular identification. One or more nano-tags can give rise to a nano-barcode, which can be used to encode information. Molecular nano-barcodes can be attached to any parent molecule, such as DNA, to code for molecular information which is not readily available from the parent molecule alone. The present invention describes nano-tag features and their application as nano-barcodes to identify individual molecules as well as score events from an assay. The present invention discloses utilizing physically or chemically distinguishable nano-tags as informational units of a nano-barcode to uniquely label a large number of compounds, particularly DNA probes, for use in detection and scoring of DNA sequence.

Current molecular labeling techniques rarely codes for information other than identifying the presence or absence of a molecule of interest. Nano-tags can be arranged into a consecutive chain to form a nano-barcode. Nano-barcodes can be prepared from one or more nano-tag units possessing properties sufficiently different from one another that they can be discriminated by the reading instrument. Variations in the tag element arrangement can give rise to a plethora of encoding schemas and informational encoding. In the present invention, new encoding schemas of nano-barcodes are disclosed to equalize their masses and provide redundant information through control codes to assure accurate readout.

Synthesis of nano-barcodes depends on the type and number of nano-tag encoding elements as well as the degree of nano-barcode complexity required. Traditional synthetic techniques make one specific product per scheme. The present invention provides an advanced mix and divide combinatorial approach, especially in the case wherein thousands or more uniquely identifiable nano-barcodes are desired. Furthermore, a bi-directional mix and divide process allows simultaneous synthesis of both the nano-barcodes and parent molecules such as DNA hybridization probes, thereby circumventing traditional probe synthesis followed by additional labeling steps.

Recent advances in the field of combinatorial sciences have identified short polymer sequences with high affinity and specificity to a given target. For example, SELEX technology has been used to identify DNA and RNA aptamers with binding properties that rival mammalian antibodies. Furthermore, phage display and antibodies or antibody fragments which bind to a myriad of compounds have been utilized to discover new peptide sequences with very favorable binding properties. In each case, a loop structure is often involved with providing the desired binding attributes. For example, aptamers often contain hairpin loops created from short regions of non-complimentary base pairing, naturally-derived antibodies utilize combinatorial arrangements of looped hyper-variable regions, and phage display libraries utilizing cyclic peptides show improved binding when compared to linear peptide phage display. The present invention utilizes molecular evolution techniques to isolate ligands specific for individual polymer subunits or group of subunits. These ligands are incorporated into the nano-tag or nano-barcode process of the present invention for detection of individual complex formation.

Although individual encoding of a parent molecule is currently possible, individual decoding is unknown. Even in the example of single molecule detection via a fluorescent moiety, the image is a composite of many photon emission events that are required to generate a sufficient signal for detection purposes. Current state of the art detection systems can visualize individual molecules. Recent advances in atomic force microscopy (AFM) and scanning tunneling electron microscopy (SEM) have made manipulation and identification of individual atoms possible. Currently, however, such applications are limited to observing individual molecules and their structures. Even though the current state of the art resolution limits can directly observe molecules and molecular units, such as nucleotides in DNA, the “reading speed” (the speed of direct molecular detection, observation, or identification) is sufficiently slow to limit this approach for most applications. When resolution is less critical, however, reading speed is increased to reasonable levels. The present invention solves this limitation by using more easily distinguishable nano-tags, or strings of nano-tags to create nano-barcodes, in conjunction with these imaging technologies. Thus, by employing readily discernable nano-tags, the resolution requirements are reduced and the speed of reading is increased to a practical rate for most biotechnology applications. In addition, this allows for the detection of individually tagged molecules in order to decipher information encoded within the molecular structure of the nano-tags and/or nano-barcodes.

Furthermore, arrays and microparticles have chemical synthesis requirements that limit the use of combinatorial chemistry procedures such as mix-and-divide type synthesis. As a result, the potential for encoding millions of individual particles is greatly complicated by the need to handle each reaction individually. The present invention provides a solution to these limitations by the application of nano-tags and/or nano-barcodes to identify individual molecules as well as track their involvement in a specific reaction or assay (see also Drmanac et al., U.S. Pat. Nos. 5,492,806; 5,667,972; 6,018,041; 5,525,464; 5,695,940; 5,882,930; and International Patent Publication No. WO 98/31836, all of which are herein incorporated by reference in their entirety). The use of nano-tags and/or nano-barcodes also permits obtaining additional information about a molecule of interest in addition to its involvement in a specific reaction or assay through the information encoded within the observed molecular structure of a nano-tag or nano-barcode.

Thus, the present invention discloses new types of nano-tags, nano-barcodes, synthesis methods, encoding schemas and reading methods or devices for more accurate and faster identification of sequences of DNA or other nucleic acid samples, analysis of other biological molecules and/or other industrial applications.

### 3. SUMMARY OF THE INVENTION

One embodiment of the present invention is directed to the encoding and decoding of information on the individual molecule level using nano-tags and nano-barcodes. More specifically, one embodiment of the present invention uses nano-tags alone or as informational units/elements of a nano-barcode, to uniquely label large numbers of compounds, particularly DNA probes, for use in detecting, identifying, and scoring of molecules, reactions, assays, and other events, such as DNA sequences in a hybridization assay.

Another embodiment of the present invention uses physically or chemically distinguishable molecular structures as nano-tags and nano-barcode elements. One aspect of this embodiment is directed to new types of nano-tags.

The present invention provides for using molecular structures including but not limited to, fullerenes, polyhedral oligomeric silsesquioxane (POSS), organo-metallic molecules, zeolite, nano-crystals, nano-tubes, and any other molecule with unique physical, structural or chemical properties, or combination thereof, as nano-tag elements. The present invention also provides for imaging techniques, including but not limited to, atomic force microscopy (AFM) and scanning tunneling electron microscopy (STM). The present invention also provides for methods of detection of differences in electron density, for example for the metal centers of organo-metallic compounds or Lathanoid-caged fullerenes. Additionally, the present invention provides for using chemical properties such as charge to provide detectable differences for use as a nano-tag. For example, aminolated POSS (+8 charge), carboxylated POSS (−8 charge), or methylated POSS (no charge). Also provided are detection devices for metallic or charged molecules, including nano-electrodes and STM. In yet a further embodiment, the absence of a tag is used as an informational nano-tag unit since the absence of a tag may be detectable via the aforementioned methods.

A further embodiment of the present invention includes any combination of one or more nano-tags to create nano-barcodes. Each nano-barcode represents one of a multitude of different nano-tag combinations. By controlling the barcode length and nano-tag positions relative to each other, many nano-barcodes can be created from only a few nano-tags. Yet a further embodiment relates to the mixing of different families of nano-tags, such as POSS, fullerenes, and organo-metallics, in a single barcode. The present invention provides for attaching nano-tags for making nano-barcodes to a nucleic acid, protein, polysaccharide or other backbone molecule, or attached to a monomer binding ligand, or having two functional groups that provide directional coupling of a single unit at one time.

Another embodiment of the invention is to synthesize, modify or tag parent (target) molecules or probe properties with nano-tags or nano-barcodes though direct labeling, subunit-specific modifications, or bi-directional synthesis. The present invention provides for attaching nano-tags to the parent molecule or probes either internally or terminally. Internal attachment can be achieved by direct chemical modification of the individual subunits of the target molecule. In this scheme, the target is modified such that individual polymer subunits can be observed through the presence or absence of the nano-tags. Yet a further embodiment provides for internal tagging through specific high affinity binding of a secondary molecule to individual subunits within the target molecule of interest. These specific high affinity molecules, or subunit detectors (SDs), may be nano-tags themselves or may have tagging elements incorporated in their structure to facilitate observation.

The present invention provides for terminal attachment through simultaneous synthesis of a probe molecule and the nano-barcode, or by attaching the barcode to the probe or object of interest post-synthesis. Such tagged probe molecules are comprised of hybrids in which one end of the molecule comprises a probe, such as a DNA-based SBH probe, and the other end comprises a nano-tag or nano-barcode. The present invention provides for combining combinatorial mix-and-divide synthesis with bi-directional synthesis to create all possible polymer sequence variations with unique nano-barcodes specific for each sequence. Such bi-directional combinatorial chemistry greatly simplifies the production of potentially millions of distinctly tagged probes through a heterogeneous preparation, as compared to current techniques of individual probe synthesis and subsequent tagging.

A further embodiment of the invention discloses the application of nano-tags and nano-barcodes as a detection method for SBH. A preferred embodiment uses nano-tags and nano-barcodes to sequence individual DNA and RNA molecules. This can be achieved via specific tagging of each SBH probe with a detectable nano-tag or nano-barcode. In one embodiment, SBH can be complemented with nano-barcode technology giving rise to detection of individual ligation products. In this embodiment, nano-barcodes are attached terminally to DNA probes to identify the nucleic acid sequence of the probe and thus the complementary sequence of the target DNA. In yet a further embodiment, the nano-tagged SBH probes can be used with an SBH assembly process (Drmanac et al., supra.) to identify and score ligated probe sequences. The application of nano-tags or nano-barcodes as DNA sequencing tools allows for the acquisition of sequence data with only a single copy of the DNA target.

The present invention also provides for nano-barcode encoding schemes. These include encoding schemes that help equalize the mass of the nano-barcodes and provide redundant information through control codes to assure an accurate readout. Encoding methods of the invention include methods wherein a symbol is assigned to a specific sequence in one or more dimensions. The encoding strings can comprise one, two or more distinct tags, or a combination of one or more distinct tags and one or more distinct spacers/linkers/backbone molecule without an attached tag. Strings of encoding nano-tags or nano-barcodes may have an equal or different number of elements (i.e. nano-tags), equal or different mass, length, or other property to distinguish them from non-coding strings. Non-coding strings read out-of-frame, whereas encoding strings read in-frame and may or may not comprise a spacer between encoding strings.

A further embodiment provides methods of verifying the accuracy of the information encoded in a nano-barcode through the use of checking systems including, but not limited to a cyclic redundancy check (CRC), frame check, or sum check, or any combination thereof.

Another embodiment of the present invention uses, but is not limited to, encoding schemes using two elements at a base four to allow a total of six (i.e. 1100, 0011, 1010, 0101, 0110, and 1001) mass-balanced codes, which are detected in-frame and may have internal frame check controls or controls based on a type of sum check. Other encoding schemas include using three, four or more elements. For SBH purposes, mass variation between tags may be utilized to weaken strong hybrids and strengthen weak hybrids by choosing the appropriate large or small tag, respectively.

The encoding schema of the present invention yields sets of distinct nano-barcodes of equal mass made of two or more nano-tags of different mass, or two or more nano-tags of equal mass but different properties. In addition, sets of distinct nano-barcodes of equal length made of two or more nano-tags of different length, or made or two or more nano-tags of equal length but different properties may be created. Furthermore, a nano-barcode or a set of nano-barcodes comprising not more than two consecutive 0s or 1s are created by using codes such as 1010, 0101, 1001, and 0110. Another embodiment of the invention comprises sets of nano-barcodes encoding a string of four symbols using two distinct tags, designated 0 and 1, each symbol encoded by singles (i.e. 1010, 0101, 1001, and 0110), duplicates (i.e. 11001100, 00110011, 11000011, and 00111100), triplicates, or larger plex.

Another embodiment of the invention provides methods to create polymer subunit building blocks from nano-tags for automated polymerization of nano-tags into nano-barcodes. For example, solid phase DNA phosphoramidite chemistry can be used to modify a bi-functionalized tag element at opposite ends, or alternatively to attach the tag element to a ribose moiety at the 1′ position in place of the traditional purine or pyrimidine base. In the former case, the tag serves as the barcode backbone, herein denoted as “tag-mediated barcodes.” The later method utilizes the phosphodiester backbone of DNA. In yet further embodiments, herein denoted “backbone-mediated nano-barcodes,” control polymerization, such as polypeptide or polysaccharide synthesis, that comprises a backbone and various side groups (or nano-tags) can be used to link nano-tags into a nano-barcode.

In another embodiment of the invention, a specifically structured nano-particle is created with an exact incorporation of several dyes, fluorophores, or other detectable moieties at various ratios or concentrations, which are then detected through discreet properties endowed by the incorporated moieties. One approach is to use dendrimers to attach a predefined number of encoding molecules. Another embodiment takes advantage of controlled polymer assembly, similar to but not limited to oligonucleotide or polypeptide chemistry. In this example, the polymer side groups are replaced with the unique property-containing moieties. In essence, these nano-particles function as three-dimensional nano-barcodes, as compared to the linear arrangement of tags previously described as nano-barcodes. These nano-particles, like nano-tags and nano-barcodes, may be used to label individual molecules or probes.

Another aspect of the present invention also includes application of nano-tags or nano-barcodes to uniquely label individual polymer subunits for polymer sequencing or for the creation of nano-barcodes. One approach is to develop nano-tagged chemical modifying reagents (i.e. for covalent attachment) specific for each type of subunit in a polymer. Another application utilizes a ligand molecule that has high affinity and specificity for individual polymer subunits (subunit detectors or SDs). For sequencing DNA, oligonucleotide detectors (ONDs) can be used and may be attached to a nano-tag. In these applications, the parent molecule is decorated with specific tags determined by the underlying subunit sequence, and the series of nano-tags becomes an informational nano-barcode using the target as a template for barcode assembly resulting in a template-mediated barcode. The newly formed barcode may remain on the original target or the individual nano-tags may be attached to each other to make a nano-barcode and then be removed from the target.

Another embodiment of the present invention includes reading methods and devices based on nano-devices. One embodiment uses a ring structure through which the nano-tags move. As the nano-tag passes through the ring, it alters the electric potential of the ring affecting electron current or causing a detectable conformational change. Another embodiment utilizes a nano-sensing lever which is either physically displaced, undergoes conformational change or measures electrical properties due to the structure or properties of the nano-tag or nano-barcode. Such devices may be modifications of existing AFM or STM devices.

One embodiment of the present invention for DNA tagging schema, utilizes the absence of modification or nano-tag as a positive informational event encoding for a particular base. Thus, the utilization of three tags and one no-tag event (a base four code) can code for each of the DNA bases (A, T, G, and C). This concept is herein referred to as the “missing tag principle.” Another application of the missing tag principle includes procedures to sequence DNA and/or RNA at a single, or few, molecule level by specifically removing a particular type of purine or pyrimidine base and then detecting the position and distance of the missing base relative to the remaining sequence. This can be done for each of the four natural bases in individual reactions followed by DNA sequence assembly from the knowledge of the missing base(s).

In another embodiment of the invention, procedures to increase the distance of neighboring DNA or RNA bases are used to open the ribose sugar moiety between the C3′ and C4′ bond and/or inserting a spacer anywhere in the phosphodiester backbone or in the open sugar molecule to facilitate the detection of molecules including, but not limited to, natural bases, modified bases, nano-tagged bases, OND-bound bases, or sites at which the base has been removed for the purposes of sequencing. Similar approaches can be applied to increase the distance of monomers in other polymers including polypeptides, proteins, and polysaccharides.

In a further embodiment of the present invention, nano-tag and nano-barcode attachment and detection is applied to any molecule, polymer, or other object for identification purposes. One embodiment comprises DNA as a molecular polymer, however nano-tag and nano-barcode technology is applicable to any molecule, polymer or object. Indeed, individual molecular coding and decoding can be used to store any information, not just information about molecular identity. In this capacity, nano-tags and nano-barcodes become a labeling system or information storage device on the molecular level. Even macroscopic objects from every day life can be labeled with nano-barcodes that are invisible to the naked eye. Yet a further embodiment of the invention is directed to encoding information in fields other than bio-detection using nano-barcodes and nano-tags.

Other embodiments of the present invention are related to various embodiments of the inventions disclosed in concurrently filed (on Dec. 20, 2002) and co-owned Provisional U.S. Patent Application Nos. 60/435,543 and 60/435,539, both of which are herein incorporated by reference in their entirety.

### 4. BRIEF DESCRIPTION OF THE DRAWINGS

FIG. 1 depicts a single round of bi-directional synthesis of DNA-based SBH probe molecules and their corresponding nano-tags. Multiple rounds can be used to create probes of desired length. This process can be used to create one tagged probe at a time or combined with mix-and-divide synthesis to create a family of tagged probes.

FIG. 2 depicts a generalized mix-and-divide process using the aforementioned bi-directional tagged probe synthesis. Two rounds are depicted in this figure. The process can be cycled as many times as necessary to create tagged probes of desired length.

FIG. 3 shows a more detailed description of the bi-directional synthesis of tagged SBH probes. The abbreviation DMTr is used for the acid-labile dimethoxytrityl protecting group and MTB for the photo-labile methyl-6-nitropiperonyloxycarbonyl protecting group. Different sized diamonds are used to symbolize the four nano-tags that represent the four bases of DNA. The nano-tag building block shown here is an example of a backbone-mediated subunit. In this scheme, the nano-tags are positioned terminally at the 3′ end of the DNA backbone. In this figure, one round of a multiple round synthesis is shown. At the bottom of the figure is a drawing of a complete tagged SBH probe.

FIG. 4 shows a more detailed description of the bi-directional synthesis of tagged SBH probes. Once again, the abbreviation DMTr is used for the acid-labile dimethoxytrityl protecting group and MTB for the photo-labile methyl-6-nitropiperonyloxycarbonyl protecting group. Different sized diamonds are used to symbolize the four nano-tags that represent the four bases of DNA. The nano-tag building block shown here is an example of a backbone-mediated subunit. Note that in this scheme the nano-tags are positioned terminally at the 5′ end of the DNA probe (compare to the 3′ orientation in FIG. 3). One round of a multiple round synthesis is shown. At the bottom of the figure is a drawing of a complete tagged SBH probe.

FIG. 5 shows a nano-tag mediated synthesis of a nano-barcode made from the organo-metallic tag element iron II phthalocyanine. The iron II phthalocyanine has been bi-functionalized to allow controlled step-wise solid phase synthesis. This scheme shows one of many potential repeating cycles. In each cycle, different nano-tag building blocks can be used to assign encoding information to each unique nano-barcode structure.

### 5. DETAILED DESCRIPTION OF THE INVENTION

The unique materials related to this invention include: nano-tags, nano-barcodes, encoded DNA probes, nano-detection devices, subunit detectors (SDs), oligonucleotide detectors (ONDs), and nano-tagged ONDs. Methods of the invention include: nano-barcode encoding and decoding schemes, utilization of the target molecule to create nano-barcodes from internal nano-tags, approaches to generating ONDs, encoding ONDs, target or backbone modifications and the use of an “empty” site as an informational unit. Their descriptions and applications are described infra.

5.1 Nano-Tags

A nano-tag comprises any nanometer-scale molecular structure that possesses chemical, structural, or physical features, properties, or attributes that affords facile detection or identification of individual molecules. Preferably, the molecules used as nano-tags range from 0.3 nm to 30 nm in diameter, and more preferably from 0.5 to 10 nm. Nano-tags may be smaller (one or a few atoms) when they have proper detectable properties. Nano-tag features that allow detection include, but are not limited to, electrochemical properties, molecular weight, color, mass, density, size, electron density, charge, shape, orientation, order, the presence of specific chemically modified functional groups, and the like, and combinations thereof. Almost any molecule with a discernibly unique physical, structural, chemical or combination of properties could be used as a nano-tag. The ability to uniquely identify individual molecules or small associated groups of molecules through imaging technology or other direct physical sensing techniques is what distinguishes a molecule as a nano-tag from other molecular tagging systems.

Nano-tags may encode at least 4, or 5, or 6, or 7, or 8, or 9, or 10, or 11, or 12, or 13, or 14, or 15, or more bits of information. Such tags are preferably detectable when fewer than 100, or 75, or 50, or 40, or 30, or 20, or 10, or 5 nano-tags of identical chemical structure are present. In contrast, current systems require thousands or even millions of tags of identical structure to be present in order to detect tags.

In one embodiment, nano-tags have properties such that two different molecules labeled with nano-tags are detected when the molecules are spatially separated by less than 100, or 75, or 50, or 40, or 30, or 20, or 10 nm.

Several molecular families have been identified as excellent candidates for nano-tags. These include, but are not limited to, modified nucleotide bases, caged metal complexes, polyhedral oligomeric silsesquioxane (POSS), octakispentacyclooctasiloxane molecules, organo-metallic compounds, subunit detectors (SDs), nano-tubes, zeolites, nano-crystals, dendrimers, nano-sized metallic particles including gold, polypeptides, and the like. Preferred nano-tag elements include, but are not limited to fullerenes, including lanthanoid caged fullerenes, organo-metallics, and POSS molecules.

Many different types of fullerenes are currently available from BuckyUSA (Houston, Tex.). Different fullerenes can have very different properties. For example, C80 is a nearly symmetric molecular ball made of carbon atoms and hydrogen, wherein the C80 refers to the number of carbons in each molecule (80 carbon atoms in C80). C60La is not only smaller (only 60 carbons), but has a metallic lanthanum center. Using the nano-detection systems described herein, there are detectable differences in both size (i.e. a sphere made of 60 carbons vs. 80 carbons), as well as electron density (hollow vs. lanthanum center) between C80 and C60La.

Organo-metallic compounds are part of a large family of molecules with a wide range of chemical properties. Members of this family of compounds have both an organic ligand moiety and a metal center. Typical metal centers include, but are not limited to the elements, chromium (Cr), iron (Fe), aluminum (Al), boron (B), cobalt (Co), magnesium (Mg), calcium (Ca), manganese (Mn), nickel (Ni), zirconium (Zr), copper (Cu), zinc (Zn), and ruthenium (Ru). Organic ligand moieties include, but are not limited to, sepulcrates, bipyridines, porphrines, corrins, ethylenediaminetetraacetic acid (EDTA), biphenyl, benzene, phthalocyanine, hematoporphyrin, heme, naphthalocyanine, cyclopentadiene, indene, fluorene, benzoindene, 4-fluorophenyl, 4-methoxyphenyl, tris(4-chlorophenyl), and the like. Most combinations of organic ligand moiety and metal center are commercially available, for example, iron phthalocyanine, ruthenium bipyridines, and cobalt sepulcrate. Clearly organo-metallic compounds have a huge combinatorial arrangement of organic and metallic moieties which gives rise to a large number of nano-tags that vary in size, shape, mass, volume, charge, and/or electron densities.

POSS molecules are semi-cubical/spherical molecular structures consisting of the element silicon (Si) and oxygen and have a variety of exterior functional groups. Currently available from Hybrid Plastics (Fountain Valley, Calif.) are amino POSS (8 amino groups with a +8 charge), methyl POSS (with 8 methyl groups and no charge), and POSS hydrate (8 negatively charged hydroxyl groups). Although the molecules are similar in physical dimensions, their electron density and charge differences can be discriminated using the detection methods of the present invention.

Based on the aforementioned tag features and detection platforms, nano-tags can be grouped into the following categories:

1) tags detected by mass, weight, density, size, electron density, orientation, order or functional groups via imaging technologies including, but not limited to: chemically modified bases, nano-structures such as fullerenes, modified fullerenes, POSS, chemically modified POSS, nanotubes, caged metals, organo-metallics, nano-crystals, nano-particles formed from polymers, and doped nano-particles;

2) tags detected by variation in potentials across a nano-pore or nano-electrode, including variations due to physical or chemical attributes including but not limited to: fullerenes, metal-doped fullerenes, POSS, chemically modified POSS, caged metals, organo-metallics, nano-crystals, nano-tubes, nano-particles formed from polymers, and doped nano-particles; and

3) tags detected by flow past a suitable detection system to detect either mass, charge, size, elemental composition, functional groups and/or density, including but not limited to: chemically modified bases, nano-structures such as fullerenes, modified fullerenes, POSS, chemically modified POSS, caged metals, organo-metallics, nano-crystals, nano-tubes, nano-particles formed from polymers, and doped nano-particles.

Additional tag element feature requirements include: a) molecular size and characteristics that do not interfere with chemical or physical functionality of the target or other tags, including but not limited to the ability of DNA to hybridize; b) unique and distinguishable physical or chemical aforementioned properties that allow two to four tags to be distinguished from one another; c) terminal tags for oligonucleotides and other compounds can be much larger in mass or size (determined by the strength of binding between the tag and target) than the oligonucleotide probe, especially if attached via a long linker; d) the number of tags needed for terminal coding of DNA probes does not exceed one tag for each type of nucleoside base in a nucleic acid chain. Thus, there is no need to encode more than the four standard bases and a few modifications such as methyl-C, unless one tag can code for more than one nucleoside. Conversely, if less than four unique tags can be detected, then a combination of two or more tags would code for a single nucleoside. Hence, one option is to have all distinct tags, or alternatively, to make codes by combining two or more tag elements.

5.2 Nano-Barcodes

One or more nano-tag elements can be arranged into a consecutive chain to form a nano-barcode (Drmanac et al., supra). Nano-barcodes are prepared from one or more nano-tag units possessing properties sufficiently different from one another that they can be discriminated by the reading instrument or detector. In one embodiment of the present invention, nano-tags, nano-barcodes, and their detection are applied to any molecule or polymer for identification purpose. Indeed, individual molecular coding and decoding can be used to store any information, not just information about molecular identity. In this capacity, nano-tags and nano-barcodes become a labeling system or information storage device on the molecular level. Nano-barcodes can also be used for the purpose of sequencing polymer subunits, in which case, the tags that make up the nano-barcode represent a contiguous series of subunits within a polymer molecule. These barcodes can be placed terminally to a DNA probe for SBH-like reactions, or be created by attaching the tags internally onto a target sequence. In either case, the actual sequence of the target polymer is represented in the informational coding of the nano-barcode.

Each nano-barcode represents one of a multitude of different nano-tag combinations. By controlling the barcode length and nano-tag positions relative to each other, many nano-barcodes can be created from only a few nano-tags. The invention also provides for mixing different families of nano-tags such as POSS, fullerenes and organo-metallics in a single barcode. Thus, one distinctive property of nano-tags is their ability to be combined with other nano-tags to give rise to nano-barcodes. Such informational multiplexing has not yet been achieved with the previously described spectroscopic techniques.

In principle, there are three approaches to creating nano-barcodes from nano-tags. The first utilizes a polymer synthesis approach in which the nano-barcode is assembled through a repeating backbone, such as the phosphodiester backbone of DNA or the amide backbone of proteins, and the nano-tags are present as side chain elements of individual polymer units (backbone-mediated nano-barcode assembly). The second approach utilizes the nano-tag itself as both an informational polymer subunit as well as the backbone of the nano-barcode polymer (nano-tag-mediated assembly). This requires that the nano-tag is bi-functional. The third approach uses nano-tags to decorate individual units of a sample target (target-mediated nano-barcode assembly). The nano-tags are assembled into a nano-barcode relative to the sequence of the target subunit. In this case, the sample target is utilized as part of the actual nano-barcode and provides order to the nano-tag elements.

5.3 Assembly of Nano-Tags into Nano-Barcodes

Once nano-tag elements are converted into polymer building blocks, they can be used in standard automated synthesizers. One approach is to assemble a series of tag elements into a specific nano-barcode and then terminally attach the complete barcode to a molecule of interest, such as DNA. This approach is limited by the laborious nature of individual homogeneous synthesis. Advantages include the ability to inspect and verify the product.

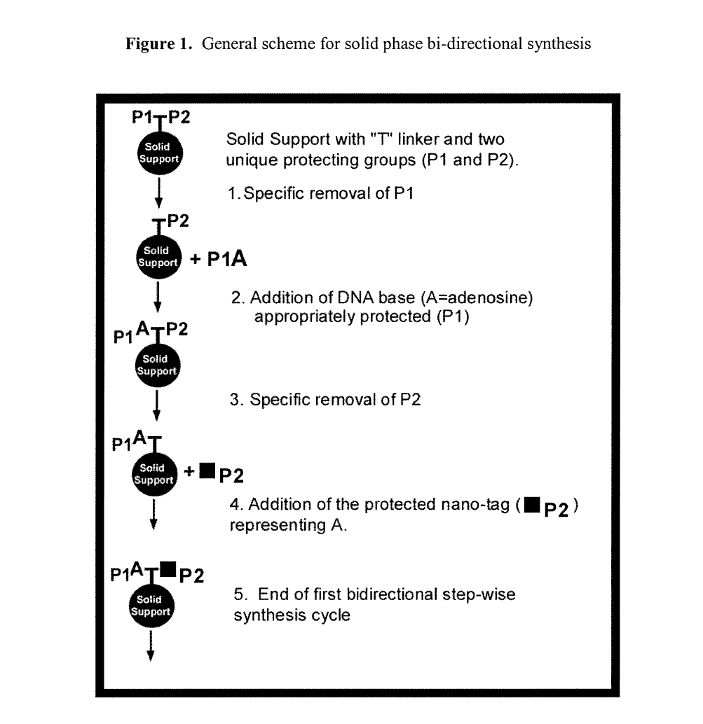

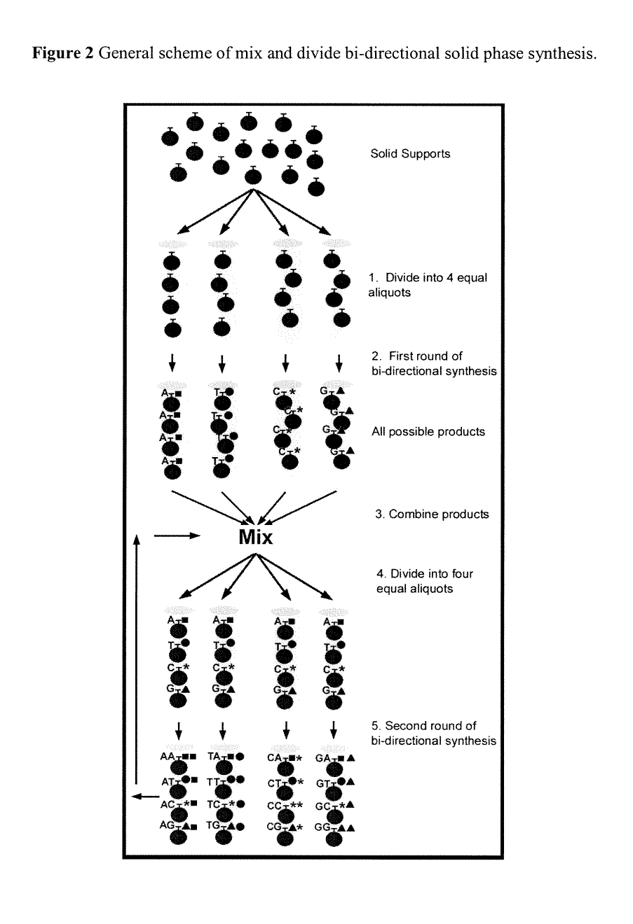

In the case where a heterogeneous mixture of terminally tagged probe products is desired, mix-and-divide combinatorial techniques may be utilized. The application of DNA sequencing of the present invention using terminally nano-encoded DNA probes in an SBH reaction, it is preferable that every possible sequence combination of DNA for a particular length (e.g. a DNA probe 5 bases in length would have 1024 possible sequence combinations) is synthesized. Each sequence is specifically identified by a unique nano-barcode. A bi-directional mix-and-divide synthesis can be used to achieve a mixture of all possible sequences of DNA uniquely defined by a specific nano-barcode for each sequence. The following example outlines one possible approach. FIG. 1 describes a single elongation cycle and FIG. 2 illustrates the use of these cycles in the context of a mix-and-divide combinatorial synthetic scheme.

The bi-directional synthesis is conducted on a solid support. Attached to the support are bi-functionalized linkers in the form of a “T” structure (FIG. 1 only shows one T structure for clarity). The two arms of the “T” are chemically protected. One arm (i.e. the “left” arm) would be compatible with the synthesis and protection scheme (P1) of the DNA molecule, and the other arm (i.e. the “right” arm) would be compatible and specific with the nano-barcode synthesis and protection scheme (P2). The two protection schemes, P1 and P2, would be unique, specific, and compatible. First, the “left arm” is specifically deprotected, and a specific DNA base is added to the deprotected terminus (in this case A or adenosine). After attachment of the base, the other protecting group (P2) is specifically removed and a nano-tag representing the base previously added is added to the deprotected terminus. Therefore, in this example, for any base that is added to one end of the bi-directional polymer, a representative nano-tag is added to the other end. Consecutive rounds of this process allows for the synthesis of polymers of the desired length. This process can be done individually for each sequence of interest, or combined with combinatorial techniques such as mix-and-divide synthesis to create entire families of bi-directional polymers.

In mix-and-divide applications of bi-directional solid phase synthesis, a family of is molecules is generated. For example, in the case of DNA, there are four possible bases that could be incorporated into the first site. In this example, the addition of A and its complimentary nano-tag are shown. In theory, A, T, G, or C could be incorporated, each with its own respective and unique nano-tag.

Mix-and-divide synthesis is used to incorporate each possible base and its respective nano-tag at each position of the growing polymer chain. FIG. 2 shows the initial population of solid support beads equally divided into four reactions. Each of the four reactions is specific for the addition of A, T, C, or G. Once the appropriate DNA base and its complimentary nano-tag are added, the supports from the four reactions are combined, mixed, and re-divided into four equal aliquots. The four aliquots are exposed to one of four specific reactions in a second cycle of bi-directional synthesis. The mix-and-divide scheme guarantees that a specific reaction is carried out with any one individual bead, yet each bead has an equal chance to be elongated by any one of the four possible building units at a given time. With sufficient numbers of solid support beads, all possible combinations can be created. FIG. 2 shows two rounds of mix-and-divide bi-directional synthesis, although protecting groups/steps are not specifically shown.

In the example depicted in FIG. 2, the mix-and-divide bi-directional solid phase synthesis of all possible dinucleotides is shown. Subjecting the library to additional rounds of mix-and-divide bi-directional synthesis creates longer bi-directional polymers. The symbols square, circle, asterisk, and triangle are used to represent the nano-tags corresponding to A, T, C, and G, respectively. When two or more nano-tags are linked together, they create/represent a nano-barcode. In the example above, the nano-barcode “square-circle” would represent the dinucleotide TA. To code for TAATA, the tags “square-circle-circle-square-circle” would be used to create the appropriate nano-barcode.

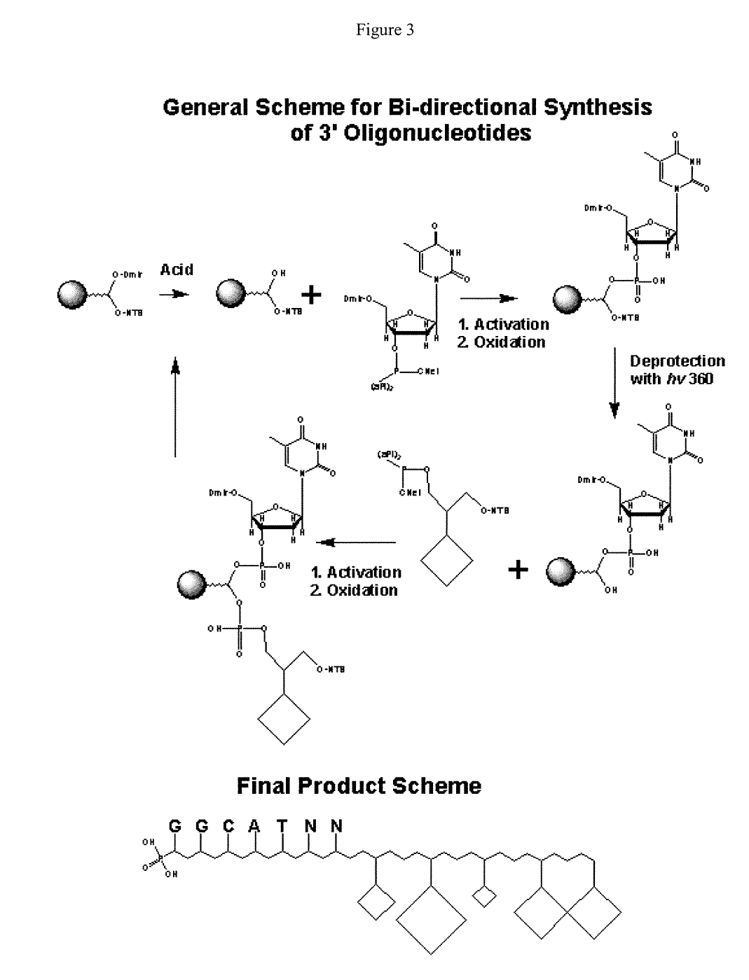

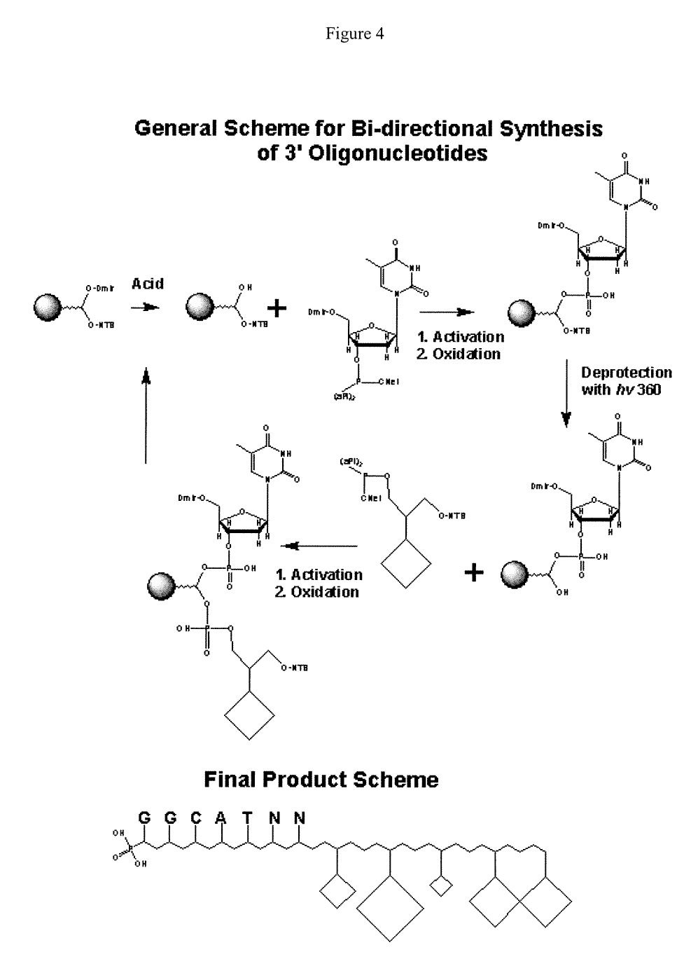

Originally, DNA synthesis was designed to be 3′ to 5′ (FIG. 3). Current advances in the state of the art DNA synthesis allows for 5′ to 3′ synthesis (FIG. 4). Thus, bi-directional DNA probes can be made with nano-barcodes in both orientations, a requirement for SBH applications of nano-barcoded probes.

FIGS. 3 and 4 show more detailed chemistry of a single round of the synthesis depicted in FIG. 1. At the bottom of each figure, the final product for SBH probes after several rounds of synthesis is shown. In the proposed scheme, the acid-labile DMTr protecting group is used for the nucleotides and the photo-labile MTB protecting group is used for nano-tags. Nano-tag elements are represented as diamonds. FIG. 3 shows the 3′ to 5′ synthesis of DNA and barcode, whereas FIG. 4 depicts the 5′ to 3′ synthesis of DNA and related barcode. Both figures show only one round of synthesis, which is applicable for mix-and-divide or individual probe synthesis. Actual synthesis will consist of numerous rounds. It is worth noting that the synthetic schemes depicted in FIGS. 3 and 4 are examples of nano-barcodes with backbone-mediated synthesis. In the example cited herein, the nano-barcode backbone consists of a simple three carbon aliphatic chain and a phosphate to give the repeating subunit structure of (PO₃CH₂CCHRCH₂), wherein R represents any of the four different nano-tag elements assigned to A, T, C, or G, respectively, but could just as easily be some other backbone of the amide backbone of peptides or the ribo-phosphodiester backbone of DNA or RNA.

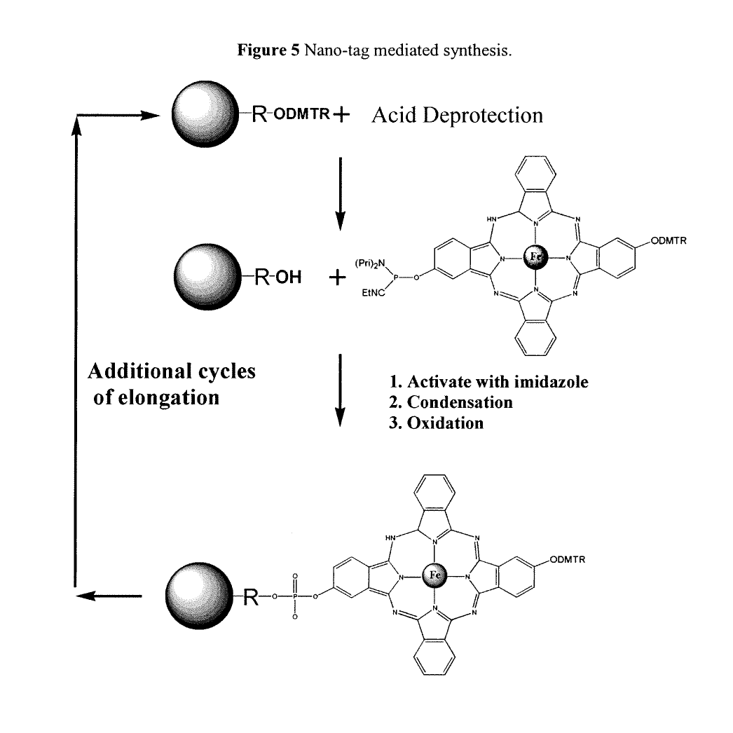

FIG. 5 depicts a nano-tag-mediated polymerization of nano-tags into nano-barcodes. Although compatible with bi-directional mix-and-divide synthesis, FIG. 5 depicts a simplified single cycle of synthesis for just the nano-barcode. Phosphoramidite chemistry is illustrated in this example, but nano-tags can also be modified with any controlled polymerization method. More details are depicted of an organo-metallic nano-tag element derived from iron II phthalocyanine (Aldrich Chemicals, St. Louis, Mo.). Phthalocyanine compounds can be purchased with many different metal centers, including Ni, Co, Ru, and others. Thus, each metal center can be assigned to encode for one of the four possible bases of DNA. Such a strategy applies to POSS, fullerenes, and other families of organo-metallic compounds.

Nano-tags can also be assembled into nano-barcodes using a template and subunit detectors (SDs). For the purposes of the present invention, oligonucleotide detectors (ONDs) are SDs designed for recognizing individual bases or an oligonucleotide sequence of two or more bases in length in DNA.

In this approach, the SDs (or ONDs for DNA applications) can be modified with a nano-tag or nano-barcode that identifies the specific affinity of the SD. The SD or OND may act as a nano-tag by itself. For example, in the application of DNA sequencing, four types of OND molecules are exposed to a DNA target. Each of the four kinds of ONDs is specific for a different DNA base. The ONDs bind specifically to their respective bases. In this manner, ONDs can recognize all possible dimers (16 in total for DNA), and even all possible n-mers (4ⁿ). Once each base or group of bases of the target is bound by an OND, the nano-tag/nano-barcode elements from the ONDs become a nano-barcode representing the sequence of the target DNA.

Recent advances in phage display library design and molecular evolution techniques have been used to develop short peptide sequences with high affinity and specificity to DNA. Wolcke and Weinhold (Nucleoside Nucleotides Nucl. Acids 20:1239-41 (2000) herein incorporated by reference in its entirety) describe an OND-like molecule that is specific for the DNA sequence TCGA. Denisov et al. (Mol. Gen. Mikrobiol. Virusol. 2:19-24 (2001) herein incorporated by reference in its entirety) demonstrate a cyclic peptide of only six amino acids that can recognize a single base difference in a DNA duplex. EPOCH Biosciences (Bothell, Wash.) is currently marketing a unique base analog constructed form pyrazolopyrimidines. This molecule is neither a purine nor a pyrimidine, but hydrogen bonds to adenine (A) with noticeably higher affinity than its natural complement, thymidine (T). By mixing pyrazolopyrimidines with single-stranded DNA (ssDNA) under appropriate conditions (high concentrations of pyrazolopyrimidines and low temperature) complexes are formed which consist of pyrazolopyrimidines and DNA in which the adenine bases have been uniquely modified by the hydrogen binding of the pyrazolopyrimidines. The DNA/pyrazolopyrimidine OND complex is imaged and the location of the adenines in the DNA polymer is thus determined.

Currently, the only high affinity nucleic acid binders available are directed specifically towards adenine. To create ONDs for C, T, and G, combinatorial synthesis and screening of the pyrazolopyrimidine molecular family can be utilized (Andres et al., Comb. Chem. High Throughput Screen. 4:191-210 (1999) herein incorporated by reference in its entirety). If necessary, the new pyrazolopyrimidine lead compounds can be subjected to combinatorial investigation and modified to increase affinity, specificity or detection ability.

A cyclic peptide phage display library can be used to pan for SD activities directed specifically towards the binding of the individual DNA bases (A, T, G, and C). Similar approaches could be used to identify ONDs with specificity for short stretches of two or more contiguous nucleotides, a base pair or continuous group of base pairs. Once identified, the OND molecules are imaged using the detection methods of the invention to determine the discrimination potential of the new OND. If necessary, the SDs are modified with nano-tag elements, thus assigning identity information to the SD.

These molecules may be further modified with stabilizers that provide stacking over the natural bases, may have a positive charge to add binding efficiency to negatively charged backbone of nucleic acids.

5.4 Creation of Nano-Tag Polymer Building Blocks from Nano-Tag Elements

The synthesis of nano-barcodes depends on the type and number of nano-tag encoding elements as well as the degree of nano-barcode complexity required. Either traditional synthetic techniques that make one specific product per scheme or mix-and-divide combinatorial approaches may be used. The latter technique is preferred in cases where thousands of uniquely identifiable nano-barcodes are desired. A bi-directional mix-and-divide process also allows for the simultaneous synthesis of both the nano-barcodes and the parent molecule such as a DNA hybridization probe. This circumvents traditional probe synthesis followed by additional labeling steps.

There are many ways of creating nano-tagged molecules and probes. One method of nano-tag creation involves mono-functionalization of a nano-tag or nano-barcode element. The mono-functionalized nano-tag/nano-barcode element is then specifically reacted with a tri-functionalized polymer backbone unit to form a unique bi-functional polymer unit. Phosphoramidite/Dmtr chemistries designed for DNA synthesis and Fmoc/tBoc chemistries developed for peptide synthesis can be used.

This method is illustrated in the following example. In this example, a mono-functionalized fullerene is used as the nano-tag/nano-barcode element, although other mono-functionalized nano-tags/nano-barcode elements can be used. The first step of this process is the preparation of a fullerene-based monomer. Mono-functionalized fullerenes are created via established chemistry to provide a unique reactive functionality, for example an amine, alcohol, carboxylic acid, and the like, at a particular position of the fullerene. This mono-functionalized fullerene intermediate is reacted with an appropriately designed tri-functionalized backbone unit. One of the functionalized positions on the backbone is specific for the tagging element (mono-functionalized fullerene in this example). The other two of the functionalized positions on the backbone are reserved for the synthesis of a polymer backbone. Standard phosphoramidite and/or dimeethoxytrityl (Dmtr) oligonucleotide chemistry and/or standard Fmoc/tBoc peptide chemistries may be used to synthesize a polymer on these two suitably functionalized positions. This chemistry is compatible with and analogous to current automated solid phase DNA and peptide synthesis techniques.

A preferred method of creating a nano-tag building block for the synthesis of nano-barcodes is via direct attachment of the necessary functional groups to create a head-to-tail polymer without a backbone. This entails a bi-functional fullerene (or nano-tag element) that has both a reactive coupling agent (such as a phosphoramidite) and a reversibly masked or protected coupling group (such as an alcohol protected with Dmtr). This approach is also compatible with current automated solid phase DNA synthesis techniques, but differs in the absence of a furanose/phosphodiester backbone.

There are many other possible ways of creating nano-tagged probes. Other methods of nano-tag creation include, but are not limited to, mono-functionalization of the tag element, which is then specifically reacted with a tri-functionalized polymer backbone unit to form a unique bi-functional polymer unit (as is the case in phosphoramidite/Dmtr chemistries designed for DNA synthesis and Fmoc/tBoc chemistries developed for peptide synthesis). Another method is the direct bi-functionalization of the tag element itself such that it can react to form a head-to-tail polymer chain (for example see Qian et al., Angew Chem. Int. Ed. Engl. 39:3133-3137 (2000); Drewry et al., Med. Res. Rev. 19:97-148 (1999), both of which are herein incorporated by reference in their entirety).

5.5 Encoding Schemas

Nano-barcodes essentially are molecular representations of abstract classification or numbering systems. As such, a nano-tag or nano-barcode consists of individual elements or molecules that can be interpreted as the symbol said element(s) or molecule(s) was designated to represent. The placement of one symbol relative to other symbols in the nano-tag or nano-barcode can also be used to encode information. For example, in the base 10 numbering system, there is a difference between the numbers 10, 01, 20, and O₂. The symbol 1 encodes for a meaning and its position relative to the symbol 0 encodes an additional meaning interpretation, or instruction. The end of a nano-barcode is established by the use of delimiting symbols or preestablished reading frames. The absence of a nano-tag, such as the use of a delimiter, or an “empty” space may also be used as an element. For example, the four element code above can include the absence of one tag element creating an “empty” space to give rise to the fourth element in a three element coding scheme.

Alternatively, a binary code can be created using a tag for one element and an “empty” space or missing tag as the other element. Encoding schemes can be based on any number of nano-tag elements. Any group of tags may encode for a single unit of information. A redundancy or check code can be integrated into the schema to limit errors.

One particular type of coding scheme utilizes a unique nano-tag for each molecule or feature in a molecule. For example, in DNA applications, one of four unique nano-tags can correspond to each of the four nucleotide bases. In the examples below, nano-tag/nano-barcode encoding schemas are in base four for the purposes of encoding the synthetic oligonucleotide, ATGCA:

FOP—F (1)

wherein F=A (adenosine), O=T (thymine), P (a POSS molecule)=G (guanine), —[a carbon chain (Cn)]=C (cytosine), is an example of a nano-bar coding scheme for ATGCA using 1 nano-tag or element per symbol in order to equalize or balance the mass of the tags across a variety of oligonucleotide sequences. Since the reading frame comprises 1 element (e.g. F), no delimeters are required. Furthermore, unique elements are used to define the base sequence.

In another embodiment of the present invention, the procedure consists of four elements, one corresponding to each base, or potentially 16 elements, one for each dinucleotide. The simplest and most preferable code for mass equilibration is a binary code, thus requiring only two uniquely identifiable elements. In this specialized binary code approach, four tag elements are used to identify a single base. For example, 1100, 0011, 0101, 1010, 1001, and 0110 all have the same mass, but code for up to six different informational units (wherein 1 may represent a tag with a larger mass and 0 may represent a tag with a smaller mass, or a spacer with no tag). This process utilizes frame checks to assure the correct orientation and thus identification of the code. One of these six codes may be used always at the beginning of a nano-tag and another at the end. Additionally, one code may be used both at the beginning and at the end and the other code as a frame control code one or more times in the middle.

Equal mass codes also help define a frame because the reading in any other frame will give many codes that do not have a total of two is and two 0s (i.e. 1000, 1011, 0111, 0100, 1111, 0000, etc.) (see example (2) below). For example, the following fullerene/organo-metallic nano-barcode encoding scheme for the oligonucleotide sequence ATGCA:

FFOO—FOFO—OOFF—OFFO—FFOO (2)

wherein FFOO=A, FOFO=T, OOFF=G, OFFO=C and —=a delimiting carbon chain (e.g. C6), uses four nano-tags per symbol (e.g. FFOO) in order to equalize the mass of the tags across a variety of oligonucleotide sequences and uses delimiters to mark the end of the nano-barcodes.

Likewise, the following fullerene/organo-metallic nano-barcode encoding scheme for the oligonucletide sequence ATGCA:

FFOOFOFOOOFFOFFOFFOO (3)

wherein FFOO=A, FOFO=T, OOFF=G, OFFO=C, uses four nano-tags per symbol in order to equalize the mass of the tags across a variety of oligonucletide sequences. However, in this example delimiters need not be utilized.

Encoding schemes can be based on any number of nano-tags/elements and any group of nano-tags (a nano-barcode) may encode for a single unit of information. For example, the following encoding scheme:

F—FF—FFF—FFFF—F (4)

wherein F=fullerene (C60), a single fullerene molecule (F)=A, two fullerene molecules (FF)=T, three fullerene molecules (FFF)=G, four fullerene molecules (FFFF)=C and —=a delimiting carbon chain (e.g. C6), is an example of using multiple fullerene nano-barcodes.

Similarly, the following encoding scheme:

FF—FO—OO—OF—FF (5)

wherein O=an organo-metallic nano-tag molecule, FF=A, FO=T, OO=G, OF=C and —=a delimiting carbon chain (e.g. C6), is an example of using mixed nano-barcodes.

Likewise, the following encoding scheme:

FFFOOOOFFF (6)

wherein O=an organo-metallic nano-tag molecule, FF=A, FO=T, OO=G, OF=C, has a reading frame comprising two elements (e.g. FF).

Additional encoding schemata can be implemented not to identify molecules, but to assure the accuracy or validity of the interpretations of the codes and limit errors. These include, but are not limited to, redundancy checks and check codes. One such schema is known as a checksum. In checksum, one region of a nano-barcode is defined as an encoded “summary” of other regions. If the summarized region is not properly manufactured or is damaged, its encoding region will not be correct. This method of error checking allows for the identification of incorrectly manufactured or damaged nano-tags and/or nano-barcodes. This process makes use of frame checks to assure the correct orientation and thus identification of the code. Such encoding schemata are well understood and need no further explanation.

5.6 SBH DNA Probes with Nano-Barcodes

DNA probes used for SBH can be terminally modified with nano-tags to create a nano-barcode representing the sequence of the probe or complimentary target. For combinatorial SBH, two universal sets of probes must be created. The first set requires the terminal tag to be 5′ of the oligonucleotide sequence and the other set requires 3′ terminal tagging.

Terminal attachment can be achieved by individual synthesis of DNA probe and related nano-barcode prior to attaching the two together. This approach is economical for smaller numbers of probes (i.e. less than 100,000 probes). A more efficient approach utilizes a stepwise bi-directional synthesis based on a mix-and-divide synthetic technique.

Mix-and-divide bi-directional synthesis creates a heterogeneous mixture of all possible DNA probe products with corresponding nano-tags. In an ideal system, all products are full-length but vary in sequence order. For DNA synthesis, one of four reactions representing the four bases is carried out for each position in the growing DNA polymer. Initially, a base-labile bi-directional linker is attached to a solid support or bead (i.e. controlled pore glass or CPG). The supports are equally divided into four reactions (one for A, T, G, and C). One terminus is specifically de-blocked (i.e. by acid or light) and the nucleotide is added using standard phosphoramidite condensation. Once the first nucleotide is added, the opposite terminus is specifically de-blocked by some other reagent than used at the opposing terminus and a specific tag representing the base on the opposite end is attached. The products of the four reactions are then combined, mixed and re-divided in four equal aliquots. A nucleic acid is once again specifically added to one end and the appropriate tag is subsequently added to the other. In this manner, any one CPG solid support will have undergone a specific process, giving rise to a population of molecules with a specific DNA and barcode sequence. Each bead, however, may follow a different or unique path due to the mix-and-divide technique and hence individual beads differ in the product sequence. This can be carried out for as many cycles as needed. The final products are combined, cleaved from the supports and all side chains are de-protected.

The key to this process is utilizing three protecting groups: reversible protecting groups for each terminus and a third for the bases. Typically, the linker and nucleosides are protected with base-labile protecting groups. The polymer terminus, typically only one terminus in standard synthesis, is protected with the acid-labile dimethoxytrityl group. This leaves the other terminus to be protected with either a highly specific group, chemical group, such as tert-butyldimethylsilyl protection and deprotection with TBAF, or a photo-labile protecting group, such as the family of nitrophenyl groups. Entire families of protecting groups and schemas are thoroughly described in Green et al., Protective Groups in Organic Synthesis, 3^{rd} ed., 1998, Wiley-Interscience, herein incorporated by reference in its entirety. In addition, all tags must be compatible with the oligonucleotide chemistry, reagents and protecting schemata.

In this manner, a base is added to one terminus followed by addition of the appropriate tagging element to the other terminus. Phorphoramidite chemistry can be controlled by the use of specific terminal blocking agents and is receptive to synthesis in both the 5′ and 3′ direction as well as 3′ to 5′ synthesis. Hence, all of the elements for synthesis of terminally-tagged and barcoded probes are present in the current state of the art automated synthesis.

By subjecting the two sets of coded probes and a sample target to SBH, a hybridization/ligation product will be formed. The ligation product will have nano-barcodes on each termini and DNA internally, and may be separated from non-ligated tagged probes. The nano-barcode will represent the sequence of the ligated probes (or complementary sequence in DNA target), thus identifying a contiguous segment of the target sequence. Nano-barcodes can be read using several methods. A preferred method of the invention is to spread them on a surface and image them by one of tip-scanning technologies, preferably STM. Negatively charged DNA or charged tags may be used to arrange nano-barcodes on a positively/negatively or hydrophilic/hydrophobic patterned surface. All possible combinations of probes will be tested to identify short contiguous sequences present within the target. From a list of short sequences present in the target, the complete sequence of the DNA target can be derived through well-established computational methods.

5.7 Internal Tagging of Polymer Subunits

Polymer subunits can be decorated with nano-tags such that the order of subunits can be derived from the corresponding order of a nano-barcode on the polymer target. Any polymer made from discreet building blocks can be “sequenced” in this manner including DNA, proteins, polysaccharides, etc. In practice, there are two methods for tagging polymer subunits: 1) direct subunit modification, and 2) specific high affinity ligand assays using subunit detectors (SDs). The first method requires a direct modification of the individual polymer subunits. In the case of DNA, this can be achieved by specific chemical modification of at least three (preferably four) bases, removal of a specific base(s) or any combination of modification and removal. Specific reactions for individual bases have been defined (Maxam and Gilbert, Meth. Enzymol. 65:499-560 (1980) herein incorporated by reference in its entirety) and other related chemistries can be developed to improve on their original efforts. The proposed modifications may be directly identified in the case where the base modification converts the base into a nano-tag element, or may require a secondary reaction specific for the modification to give rise to a detectable feature. Such a strategy is not limited to DNA, but can be applied to any polymer made from distinct subunits. Oligomers such as oligonucleotides of known sequences that contain modified monomers may represent themselves as nano-barcodes that can be used for tagging other molecules that have to be analyzed. Chemical modification of pre-made oligomers or synthesizing oligomers from modified monomers is two ways to obtain these types of nano-barcodes.

The specific high affinity approach requires isolating or developing specialized molecules (i.e. SDs) with both high specificity and high affinity for polymer subunits or short strings of polymer subunits. Such molecules can be created through molecular evolution techniques to bind any target of choice (Gold, Proc. Natl. Acad. Sci. USA 98:4825-6 (2001); Gold et al., J. Biotechnol. 74:5-13 (2000); Andres et al., Comb. Chem. High Throughput Screen. 4:191-210 (1999); Klug and Famulok, Mol. Biol. Rep. 20:97-107 (1994), all of which are herein incorporated by reference in their entirety). These SD molecules may be discriminated by one of the nano-tag detection methods of the invention or they may require attachment of a nano-tag to each binder for identification. As with the direct modification approach, the specific high-affinity binding units utilize the polymer backbone of the target to arrange the detectable elements to resemble a nano-barcode (target-mediated nano-barcodes). Monomers of known sequence may be used to produce nano-barcodes by linking nano-tags after specific binding to monomers. Oligonucleotide detectors (ONDs) or base-specific binders or detectors exemplify this approach for DNA and/or RNA sequencing and other types of analysis (see below).

5.8 Analysis of Polymers by Specific Modification of or Ligand Binding to Monomers

The present invention provides for a method for analysis of partial or complete sequencing of a polymer molecule (comprising, for example, nucleic acids, amino acids, saccharides, or other monomers) by modifying some or all monomers of one, a few, or all monomer types in a polymer consisting of two or more monomer types. Ligand binding to monomers can also be defined as modification of polymer molecules. The position(s) of a modified monomer(s) is detected in a single intact polymer molecule providing the specific type or group identity of the monomer at a position in the polymer. Relative positions of modified monomers can be determined with absolute or approximate distance to provide the sequence or signature of the analyzed polymer. In addition to monomers, short oligomers of specific sequence can be specifically modified as units. In addition to modifying monomers for enhanced speed or accuracy of monomer detection, chemical modifications can be applied to increase distance between monomers to allow detection or increase efficiency (i.e. accuracy, speed, success rate) in detecting neighboring monomers. Another embodiment of the invention provides for complete fragmentation before or after modifications to increase the distance between monomers while preserving the order of monomers. Such methods also include the unwinding of the secondary and tertiary structures of the polymer without introducing undue breaks in the chain. This can be achieved by elevating the temperature, changing the pH, by mechanical forces, and the like.

One or more unique SDs can be used to sequence multi-subunit polymers by exposing the polymer to an excess of the SDs and allowing them to associate under favorable conditions. The method can be conducted with one type of SD in a particular reaction and with different reactions conducted for each subunit type. It is also possible to simultaneously react multiple types of SDs with a target as long as the different SDs can be discriminated. The polymer is decorated with uniquely identifiable SDs in linear order corresponding to the underlying subunit sequence. The SD-polymer complex is subjected to detection to determine the sequence.

Modification can be done in several ways, including but not limited to: a) by adding a chemically bonded molecular group or structure that may carry a nano-tag or nano-barcode; b) by adding a ligand, monomer or SD that may be nano-tagged or nano-barcoded; c) by specifically removing a portion of a monomer or a complete monomer; or d) by replacing a portion of a monomer or a complete monomer with a molecular group or structure that may carry a nano-tag or nano-barcode.

For sequencing purposes, almost all monomers have to be modified and/or reading results from several molecules have to be integrated after detection in one reading result. Added molecules or particles with or without replacement have to be small enough to allow preservation of the chain and to allow detection of two close or consecutive monomers. The monomer specificity of ligands can be determined or enhanced by some prior modifications of the given monomer.

Various schemata can be used to perform specific modification of monomers belonging to certain types. Specific reagents may be used that chemically interact only with one or some but not all monomer types or subtypes, for example, only with methyl-C in DNA and not with regular C. Other methods involve the use of natural or designed molecules that demonstrate the necessary selectivity and binding efficiency. Such modifying molecules may be used alone or they may be used in combination with nano-tags and/or nano-barcodes so they may be accurately detected. Coupling with nano-tags or nano-barcodes may be accomplished either before or after modification of the monomers. Monomers that are modified by complete or partial removal may also be specifically nano-tagged and/or nano-barcoded.

Modification methods can include one reaction or multiple reactions. All necessary bases can be modified on the same nucleic acid molecule or on aliquots from the same sample in separate reactions. Separate reactions may provide more efficient identifications especially if larger tags are used. Furthermore, multiple reactions allow the use of modifications that are not specific for a single base type. The modifications of ligands may not be specific to only one type of monomer, or base in the case of nucleic acids. In one embodiment, modification of each of two monomers with one monomer in common can be used in two reactions to define three monomers. The two modifications need not be distinct between themselves, but they must be distinct from non-modified monomers. For example, in nucleic acid chemistry, one modification reaction is specific for two types of bases and a second modification is specific for one of the aforementioned modified bases plus one of the two remaining unmodified bases. Therefore, in the first reaction, a base type is either modified (+) or unmodified (−). In the second reaction, one of the two bases from the first reaction would be specifically modified as well as another base. Using this approach, all four bases are distinguishable in that they have been modified in both, only the first, only the second, or in neither reaction (i.e. +/+, +/−, −/+, and −/−).

This process requires integrating data from both reactions corresponding to the same nucleic acid segment. The integration may be done by taking a nucleic acid fragment analyzed in one reaction and then searching in the other reaction for a corresponding fragment that shares all or a significant portion of the sequence that can be recognized by size, pattern of modifications, non-modifications, nano-tags, or nano-barcodes. Such a process may rely on the presence of a complementary strand in each reaction. For example, if C and G are modified in one reaction, complementary strands will have either modification (C or G) or no modification (A or T) at the matching position.

Combinations of various types of modifications and no modifications may be used to discriminate all monomers in a polymer. For four standard base monomers in nucleic acids, one of the larger bases (A or G) may be modified by addition to be the largest, and one of the smaller bases (C or T) may be removed, and the two remaining may be discriminated by natural differences, such as two rings vs. one ring, smaller mass or dimensions, and significant differences in other physical or chemical properties. This schema may be done with only one ligand specific to A or G, and one specific chemical or enzymatic removal of C or T.

Modifications can be performed on a small amount of polymer that may be represented with only one or a few molecules. The polymer has to be in the form that allows access of reagents or ligands to monomers. Polymers may be long or fragmented chains, (i.e. chromosomal DNA). Fragmentation can be done enzymatically (i.e. endonucleases, including restriction enzymes), or mechanically (i.e. ultrasound, high pressure), or chemically (i.e. depurination). Digestion with restriction enzymes may be partial or complete. In one embodiment, individual longer polymer chains may be further fragmented to improve analysis by providing tracking of spatial or time isolation or otherwise. After modification, the polymers may be purified for a detection step that may be in solution or after attaching polymers on a surface. The surface may be prepared or patterned to allow proper spacing orientation or configuration (for example, stretching) of modified monomers, that may involve interaction with some introduced modifications. In addition, electric, mechanical, flow, magnetic, or chemical forces including microfluidics can be used for positioning or transporting polymers for the detection step.

The position(s) of a modified monomer(s) is then detected providing for the identification of a specific type or group of monomer at particular positions in a single intact polymer molecule. The positions and distances of modified monomers can be detected by various methods described herein depending on the type of modification. To provide a sequence or signature for the analyzed polymer, the detection methods must only recognize the modification of the monomer, the type of modification if different modifications are used simultaneously for different monomers, and the relative or absolute distance or position of modified monomers. Scanning tip technology with atomic or molecular resolution is a preferred way of detecting introduced modifications along the polymer chain. Multiple reads may be done on the same polymer molecule.

Analysis of the data involves translation of primary measurements into monomer type or position or order with or without distance information for each polymer molecule. It may optionally integrate the data from two or more molecules sharing the same segment, as well as integrate longer chain information or sequence from overlapping fragments.

Direct single polymer molecule analysis or sequencing allows analysis of large molecules and whole genomes or populations of polymers without subcloning. Random fragmentation of 5 to 5000 initial clone molecules, chromosomes, or genomes may be performed. Sufficient sampling of a population of individual fragments gives necessary information to assemble whole sequences. The method provides the ability to determine haplotypes by analysis of fragments longer than about 1000 bases, preferentially longer than 5000 bases, and assembly of contigs, for partial or whole parental chromosomes, by overlapping fragments that share the same allele. One approach is to modify longer fragments and if necessary, to fragment them before reading but keeping the group of short fragments belonging to a longer fragment together.

Polymer analysis may be used for diagnostics, finding rare mutations by counting molecules that have it and those that do not have it, and for determining the frequency of damaged or chemically altered bases in a cell or tissue under the influence of certain chemical, environmental or genetic instability.

Another embodiment of the present invention involves determining the expression by analyzing a population of mRNAs, cDNAs, or proteins from a tissue or a cell that involves counting the molecules representing the same gene, the same splice variant, or the same protein variant. This process does not require complete sequencing. Simply determining a unique gene/protein/variant signature is sufficient.

5.9 Base Ligand Molecules and Nano-Tags Attached to Ligand Molecules for Sequencing DNA

The present invention provides for methods of nucleic acid sequencing comprising specifically modifying one or more base types in a nucleic acid molecule and identifying base type at two or more consecutive positions in an individual intact nucleic acid molecule. Modification of only one base type may be sufficient if other bases can be differentiated without modification. One option is to modify one small and one large base, for example C and A. Base modifications may be mediated by: a) base-specific ligands that include, but are not limited to peptides, cyclic peptides, or aptamers; b) direct chemical modification by addition; c) direct chemical modification by partial or complete removal; d) chemical modification by partial or complete replacement; or e) indirect chemical modification by addition, replacement, or removal.

Another application for nano-tags includes single base detection (SBD) sequencing through the use of subunit detectors (SDs), such as oligonucleotide detectors (ONDs), which has significant advantages of speed, ease of use, and cost compared to other sequencing methods. In one embodiment of SBD sequencing, one or more single-stranded DNA molecules are exposed to a set of detectors that recognize and specifically bind to individual nucleic acid bases with high affinity. A family of four detectors, one for each base, can then be used to specifically bind and thus modify each base specifically with the appropriate binder. Thus, tagged ligands specifically bind individual subunits and are oriented in the same order as the polymer sequence. The tags are detected using scanning tunneling microscopy, atomic force microscopy, or other physical methods capable of detecting any of the aforementioned properties of nano-tags and their order on the target will represent the target sequence. It is possible that the ligand itself can act as a nano-tag or it may be necessary to add nano-tags. This process allows direct reading of nano-tags on a single molecule without the need to determine the content of oligomers in the polymer sequence. By rapidly scanning a field of individual DNA fragments, the detectors are used to identify DNA bases present in each fragment by identifying the sequence of tagged ligands or ligands themselves that bind to consecutive bases (or other monomers). By compiling the sequences of thousands of such fragments, the entire sequence of very large DNA molecules can be determined without cloning or PCR amplification.

One embodiment of sequencing DNA uses four base-specific OND molecules from a cyclic peptide phage display library. The candidate OND molecules must have high affinity and specificity for DNA bases, which can be achieved by using an appropriately designed selection scheme. Typically, selection is used to develop affinity and counter selection is used to increase specificity. After several rounds of selection and counter selection, a consensus sequence is usually identified that corresponds to a family of base-specific ONDs. In the case where a small family of consensus molecules is identified, additional molecular evolution techniques can be employed through direct chemical synthesis followed by additional rounds of selection and counter selection. Each OND, one for each natural base, will be labeled with a nano-tag to reflect which base it binds.

For sequencing purposes, a small fragment (10-100 nucleotides in length) of DNA is isolated. For assembly of large sequences of DNA from smaller fragments, more than one copy may be used, but each copy can be individually observed and scored. The DNA fragments may need backbone modifications to accommodate the binding of ONDs. This can be achieved by selectively opening the DNA's sugar moiety between the 4′ and 3′ bonds or by adding a spacer anywhere in the phosphodiester backbone.

The DNA fragments are exposed to an excess of the four ONDs under favorable association conditions and allowed to react. The ONDs will bind their respective bases and thus decorate the DNA with nano-tags corresponding to the underlying DNA sequence. In this scenario, the DNA plays an active role in nano-barcode assembly, hence the name “template-mediated” nano-barcode. The OND-modified DNA molecules are isolated and subjected to detection. The products are imaged or read by passing through a detector, each tag is observed and the DNA sequence is identified from the sequence of tags. Several small semi-overlapping fragments can be used to assemble the larger parent molecule.

Optimized SBD not only requires ligands that specifically recognize individual DNA bases with high affinity, but also requires minimal negative interaction with surrounding bases or bases with ligands. This ensures that each binding event does not hinder ligand binding to nearby bases. One solution is to lengthen the polymer backbone through backbone-modifying chemistry. Alternatively, binding might be synergistic, with each correct binding event increasing the chances of correct binding at neighboring bases. Base-specific nano-tags attached to base-specific ligands should be easily distinguished from one another and should interact minimally (or synergistically) with one another and with nearby ligands and the DNA bases of the fragment.

Recent advances in heterocyclic chemistry and cyclic phage display techniques have afforded numerous commercially available molecules with SD and OND-like properties (New England Biolabs, Beverly, Mass.). SDs, ONDs, and base-specific ligands can also be readily produced, identified, and developed through the use of combinatorial molecular evolution techniques and phage display. Combinatorial molecular evolution techniques preferably include, but are not limited to, solid phase techniques designed for DNA, peptide, and polysaccharides. The most likely molecular candidates are controlled polymers with several building block units, such as cyclic peptides, polysaccharides, protein-DNA (PNA), bi-functionalized heterocycles, decorated scaffolds, or a combination thereof.

Positive and negative selection is critical for isolating molecules that bind to the consecutive bases (or monomers in general) strongly, specifically and independently. In selecting four base binders, positive selection may be done with four oligonucleotides about 10 to 100 bases in length, such as: -aA-aA-aA-aA- . . . -aA-aA; -tT-tT-tT- . . . -tT-tT; -cC-cC-cC- . . . -cC-cC; and -gG-gG- . . . -gG-gG-, wherein -a, -t, -c, and -g denote any of the other three bases. These oligonucleotides provide for 50% of the base that is designed for, and about 17% for each other, and binding has to occur to the single positive base. Alternating positive selection may be done with: AAAA . . . AAA; TTTT . . . TTT; CCCC . . . CCC; or GGGG . . . GGG targets that are 100% made of one base. Negative selection may be done with four nucleotides: -a-a . . . -a-a; -t-t . . . -t-t; -c-c . . . -c-c; or -g-g . . . -g-g. These oligonucleotides may be biotinylated to collect them with streptavidin beads, columns, or multi-well plates.

Yet another method of the invention provides for creating four specific base binders (or more for modified bases, such as methyl-C) by designing oligopeptides using the consensus sequences (and slight variants) of the binding sites of ATP and GTP (and other) binding proteins (Vetter and Wittinghofer, Science 294:1299-1304 (2001), herein incorporated by reference in its entirety). Selection of amino acids to incorporated in base-specific peptides may be done by their reported affinity to specific bases (Luscombe, et al., Nucl. Acids Res. 29:2860-2874 (2001), herein incorporated by reference in its entirety). An additional possibility is to create short peptides that are very specific catalysts (Sculimbrene and Miller, J. Am. Chem. Soc. 123:10125-10126 (2001), herein incorporated by reference in its entirety) to modify selectively or specifically one monomer.

5.10 Direct Nano-Tagging of DNA Bases for Sequencing

A simplified approach to internal tagging is through direct modification of polymer subunits. These modifications can convert the polymer subunit into a nano-tag. Modifications developed by Maxam and Gilbert (Meth. Enzymol. 65:499-560 (1980) herein incorporated by reference in its entirety) were initially developed to specifically modify the bases to allow cleavage of the DNA backbone at the modification site. The methods of the present invention modifies bases for detection only and not for cleavage of the DNA backbone.

Since the work of Maxam and Gilbert, several additional DNA modification chemistries have been developed. Advances in heterocycle chemistry and protecting schemata for solid phase DNA synthesis have given rise to a multitude of specific modifications. The chemical differences between purines and pyrimidines are expected to be sufficient for discrimination, however two purines or two pyrimidines are too similar for easy discrimination. Thus, specific modification of at least one of the purines and one of the pyrimidines should facilitate discrimination. For example, phenoxyacetyl (Pac) or benzoyl (Bz) molecules can be used to modify the amino groups of A and C only, thereby allowing discrimination of A(Pac) from G and C(Pac) from T, respectively. It is worth noting that the modifications themselves become to discriminating factor and thus act as nano-tags in this situation. Similarly, the arrangement of modified or missing bases also forms a nano-barcode.

Certain DNA damaging agents can be considered in sequencing applications due to their ability to specifically modify a particular base or group of bases. There are many known base-specific chemical modifications including ionizing radiation, oxidation, reduction, cross-linking, methylation, and dimerization.

In yet a further embodiment of the invention, several destructive processes specific to guanine bases can be used to specifically modify (i.e. remove) all guanines. Hence, the missing bases in a DNA chain can be identified as G and all of the remaining bases are non-G. Destructive processes such as the one described herein are limited because individual reactions must be carried out for each of the four bases. This is an example of the “missing tag” schema. Additional chemistries are known that cause specific abasic sites (Lhomme et al., Biopolymers 52:65-83 (1999) herein incorporated by reference in its entirety) that may be incorporated into the missing tag schema. A preferable approach is to specifically remove all Gs from their sugars to discriminate between the two purines (A and G) and then to modify all remaining amino groups to modify C, hence discriminating C from T. This is an example of how only two reactions could specifically modify all four DNA bases to create satisfactory base-specific nano-tags.

One caveat to direct modification chemistry is the requirement to have 100% conversion with absolute specificity. The lower the reaction yields, the more unreliable the sequence information becomes. One solution to this problem provided by the present invention is to sequence several copies to find a consensus sequence that is based on enough statistical information to ensure confidence in the final result.

5.11 Target Backbone Modifications

The main constraint to SBD, ONDs and perhaps nano-tags is the distance between neighboring bases or subunits, which may cause steric hindrance. Side chains for the OND-bound nano-tags are one possibility to help decrease physical constraints or interactions between tags, probes, and targets. Furthermore, it may be possible to design nano-tags that interact with both the target molecule as well as each other, to help stabilize a macrostructure that will be read as a nano-barcode. Other possibilities include spacing the parent polymer units by altering the backbones. For DNA, cleavage of the C4′-C3′ carbon-carbon bond in the furanose moiety or insertion of a lengthening element into the phosphodiester backbone is one method to decrease stearic interference. Such modification would lengthen the distance between individual bases of DNA. Such an approach is not limited to DNA, but can be developed and applied to lengthen any polymer backbone.

5.12 Detection Devices and Sensitivity of Reading

A number of well-defined methods can be used to identify and discriminate between individual nano-tags and/or nano-barcode elements. These methods include, but are not limited to, scanning tip imaging technologies such as atomic force microscopy (AFM), scanning tunneling microscopy (STM), and other spatial imaging techniques, such as scanning electron microscopy (SEM), transmission electron microscopy (TEM), and scanning force microscopy (SFM), and related techniques; measuring potentials with nano-electrodes, and the like, and combinations thereof. These methods can be used to determine information about the nano-tag properties including, but not limited to size, color, electron transmission properties, spatial coordinates, and the like. Any individually discernable chemical or physical property of a nano-tag or nano-barcode can be utilized by the present invention.

In a preferred method, a high speed STM, AFM or other molecular visualization device is used to scan an area that contains nano-tags or nano-barcodes. In a most preferred method, multiple devices, such as an array of STM tips, are used to scan different areas of the same substrate, thus increasing the speed of data acquisition. Alternatively, multiple tips can scan the same areas thus providing a more redundant measurement. In addition, modifications to such instruments can be made in the sensory tips to optimize nano-tag detection. TEM and SEM or other molecular visualization techniques can be used to read tags.

In general, a sample is deposited onto a clean, flat surface usually made of gold or mica, but may also include grooved surfaces and molecularly patterned and/or charged surfaces. The substrate may be exposed to an energy field for orientation purposes. The sample is then subjected to visualization as a detection tip scans over the surface. Typically a current of electrons passes from the scanning tip to the surface. When the tip encounters a molecule of interest, the current is altered. Thus, two pieces of information are collected: 1) the location (or frame) of the nano-tag/element, and 2) the tunneling current of the nano-tag element. Once the substrate is scanned, an image is assembled and analyzed. The properties and position of a nano-tag relative to other nano-tags are used to determine the information content of the nano-tag.

For DNA sequencing by hybridization (SBH), complementary probe sets terminally labeled with nano-barcodes that are juxtaposed relative to each other can be ligated, for example with the enzyme T4 ligase. The ligated products are deposited on the imaging surface and then subjected to STM. The tag elements are distinct enough that a low-resolution image can give satisfactory discrimination. The information within the nano-barcodes yields the sequence of small contiguous regions within the sample. By applying SBH assembly algorithms, the small fragments can be assembled into the much larger parent molecule's entire sequence.

An additional class of detectors is provided by the present invention. Nano-scale manufacturing methods can be used to produce sensors capable of detecting tags or series of tags. One such sensing device is a loop structure that has nanometer dimensions (both diameter and thickness are in the range of 0.3 to 3 nm or 0.3 to 10 nm), and is connected to a signal reporting electrode or other tool. The nano-loop may be created by cutting a ring from a nano-tube or it may be built from molecular units, including but not limited to short cyclic polymers such as cyclic peptides. The loop can have different chemical properties or group pointing to the interior of the loop. One example is to have charged groups inside and have neutral groups facing outside. These features can enhance the sensitivity of detection because the signal change is dependent only on the internal side.

Modifications to imaging instruments, such as AFM and STM can be made in the sensory tips to optimize nano-tag detection. The present invention provides for the use of a nano-ring-like structure through which a nano-tag or nano-barcode can fit. The passing nano-tags can case physical changes by altering current, deflecting the tip, or causing a conformational change. A lever arm attached to the imaging devices can measure force exercised by conformational change or similar effects.

The nano-barcodes or natural molecules such as a DNA molecule having natural bases including methyl-C or other naturally or intentionally modified or tagged bases, are caused to pass through the loop by random movements or by a flow or other directional force. As each unit tag passes through the loop, its interaction with the loop is measured by the loop's capacitance change or otherwise. Additionally, tag properties can be measured through non-covalent interactions with structures on the loop. Furthermore, nano-tag structures can affect the ability of the loop to conduct electrons originating from the donors present in the loop. Such an effect can be measured and used to determine the presence of a nano-tag and/or the properties of the nano-tag. The resulting signal profile represents consecutive tag elements or natural bases, or modified bases, and allows barcode decoding or sequencing of DNA or other polymers. Other nano-scale schemas can be used to determine tag content. Multiple nano-loop devices can be arranged in a row or in an array for parallel analysis of tens to thousands of molecules or nano-tags.

A nano-scale device that undergoes a conformational change in the presence of a tag or molecules and can relay its conformational state to a measurement device is an alternative method of reading tags. Such a device could have multiple conformation states, each state being specific for a particular nano-tag type. Conformational changes are typical in interactions between two proteins or a protein and DNA or RNA molecule, including bending, rotating or other movements of parts of molecules under interaction forces (Schneider, Nucl. Acids Res. 29:4881-4891 (2001) herein incorporated by reference in its entirety). This phenomenon is utilized here by selecting or engineering sensors with two or more conformational states that are taken in the interaction with corresponding nano-tags or molecules in general. One option is to have a sensor with a hole with a binding site. When a nano-barcode or polymer such as DNA with natural, modified, or tagged bases, passes through the hole, each unit tag or monomer will cause a temporary conformational change before it is released and the next tag begins its interaction. Conformational changes of the sensor is detected in many ways, including but not limited to AFM wherein different forces on an AFM tip are detected (Basche et al., Proc. Natl. Acad. Sci. USA 98:10527-10528 (2001), herein incorporated by reference in its entirety). The AFM tip is connected to the sensor that may be connected to a support. Another option is a reporting molecular wire that is connected to the sensor is moved to establish contact with one specific out of two to four or more connecting wires on a switching board.

One important distinction between the present invention and current techniques is the number of molecules and events needed to obtain results. In all other known methods, literally thousands if not millions of molecules are observed as a population and their summed consensus signal gives rise to an event detectable above background. The methods and devices of the present invention provide for detection of individual molecules with a single observation. Although single molecule detection has been achieved for fluorescence, the technique requires multiple emission events for each observation.

Numerous modifications and variations in the practice of the invention are expected to occur to those skilled in the art upon consideration of the foregoing description of the presently preferred embodiments thereof. Consequently, the only limitations which should be placed upon the scope of the present invention are those which appear in the appended claims.

1. A method of analyzing a polymer comprising the steps of:
a) modifying a monomer in a polymer consisting of two or more monomer types; and
b) detecting the position of the modified monomer in a single intact polymer.

2. The method of claim 1, wherein two or more monomers of a particular type are modified.

3. The method of claim 1, wherein two or more monomers of a particular type are detected.

4. The method of claim 1, wherein different monomer types are modified.

5. The method of claim 1, wherein two or more types of monomer in a polymer are modified and detected.

6. The method of claim 1, wherein the modification is selected from the group consisting of adding at least one atom, removing at least one atom, and replacing at least one atom.

7. The method of claim 1, wherein the polymer is an oligomer.

8. The method of claim 1, wherein the polymer comprises nucleic acids.

9. The method of claim 1, wherein the polymer comprises amino acids.

10. The method of claim 1, wherein the polymer comprises saccharides.

11. The method of claim 1, wherein the detection step comprises detecting properties selected from the group consisting of charge, shape, mass, electron flow, and combinations thereof.

12. A method of detecting nano-tags comprising the steps of:
a) passing a nano-tag into a confined space which causes a conformational change in the structure of the nano-tag; and
b) detecting the conformational change of the nano-tag.

13. The method of claim 1 or 12, wherein the detection step comprises an imaging process selected from the group consisting of atomic force microscopy, transmission electron microscopy, scanning tunneling electron microscopy, and combinations thereof.

14. A method of nucleic acid sequencing comprising the steps of:
a) specifically modifying one or more base types in a nucleic acid molecule; and
b) identifying the base type at two or more consecutive positions in an individual nucleic acid molecule.

15. The method of claim 14, wherein the modifications are mediated by a base-specific ligand.

16. The method of claim 15, wherein the base-specific ligand is selected from the group consisting of a peptide or an aptamer.

17. The method of claim 14, wherein the modifications are mediated by a method selected from the group consisting of
a) direct chemical modification by addition of a polymer subunit type,
b) direct chemical modification by partial or complete removal of a polymer subunit type,
c) chemical modification by partial or complete replacement of a polymer subunit type, and
d) indirect chemical modification by addition, replacement, or removal of a polymer subunit type.

18-34. (canceled)
